(19) United States
(12) Patent Application Publication
(10) Pub. No.: US 2007/0298022 A1
(43) Pub. Date: Dec. 27, 2007

(54) Identification of Phospholipase A2 as Target in Cancer Treatment, with Special Emphasis on Colorectal Cancer and Its Mechanism of Action

(76) Inventors: Andreas Scherer, Freiburg (DE); Ruben Papoian, Cincinnati, OH (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/813,619

(22) PCT Filed: Jan. 12, 2006
(86) PCT No.: PCT/US06/00953

§ 371 Date: Jul. 10, 2007

Related U.S. Application Data
(60) Provisional application No. 60/643,990, filed on Jan. 14, 2005.

(51) Int. Cl. A61K 38/00; A61P 43/00; C12Q 1/42; C12Q 1/68

(52) U.S. Cl. 424/94.1; 435/21; 435/6

(57) ABSTRACT

A peptide of phospholipase A2 had been detected in human plasma and synthesised. The peptide was injected into mice and gene expression profiling on many organs performed. Phospholipase A2 showed significant effects on regulation of gene expression in the liver. The genes affected are members of the integrin signalling pathway, wnt pathway and PTEN pathway. The changes in gene expression indicate a positive effect of phospholipase A2 on cell proliferation and invasiveness. The gene annotation points at colorectal cancer.

### RELATED APPLICATIONS

This application claims priority to U.S. Ser. No. 60/643,990, filed Jan. 14, 2005, the contents of which are expressly incorporated by reference.

### FIELD OF THE INVENTION

This invention relates generally to the analytical testing of tissue samples in vitro, and more particularly to aspects of gene expression in cancers.

### BACKGROUND OF THE INVENTION

“Cardiovascular Disorder Plasma Polypeptides” (CPPs), fragments, and post-translationally modified species of CPPs are present at a lower level in plasma obtained from individuals with Coronary Artery Disease (CAD). CPPs are secreted factors and, as such, are readily detectable and useful for drug development, diagnosis, and prevention of cardiovascular disease. See, PCT/EP2004/007842, “SECRETED POLYPEPTIDE SPECIES REDUCED IN CARDIOVASCULAR DISORDERS” filed Jul. 15, 2004. A fragment of phospholipase A2 (PLA2) was detected in human plasma and synthesised by GeneProt, Inc. (Geneva, Switzerland). This peptide was termed GP_{—}1221076 (also CPP-51 or GPA071).

There is a need in the art for additional information about the function of phospholipase A2 and fragments thereof in human plasma.

### SUMMARY OF THE INVENTION

A secreted phospholipase A2, the GPA071 peptide, was injected into mice. Gene expression profiling was performed on many organs. Surprisingly, the GPA071 peptide showed significant effects on regulation of gene expression in the liver. The genes affected are members of the integrin signalling pathway, wnt pathway and PTEN pathway. The changes in gene expression indicate a positive effect of the GPA071 peptide, and PLA2 generally, on cell proliferation and invasiveness, the gene annotation points at colorectal cancer. To our knowledge this may be the first time that secreted phospholipase A2 has an upstream stimulatory effect on integrin signalling.

Thus, the invention thus provides the use of a polypeptide (a secreted phospholipase A2, such as a GPA071 peptide) for the manufacture of a medicament for use in the treatment of a proliferative disease or condition, such as cancer, in particular colorectal cancer.

In a further aspect, the invention relates to a method for the treatment of a proliferative disease or condition, such as cancer, in particular colorectal cancer, comprising administering an effective amount of a polypeptide as defined above to a mammal including a human suffering from the disease or condition.

In another aspect, the invention relates to a pharmaceutical composition for use in a proliferative disease or condition, such as cancer, in particular colorectal cancer, comprising an effective amount of a polypeptide as defined above and a pharmaceutically-acceptable carrier.

In another aspect, the invention relates to the use of phospholipase A2 as a therapeutic target in cancer treatment, in particular, in the treatment of colorectal cancers.

### DETAILED DESCRIPTION OF THE PREFERRED EMBODIMENTS

Using a Velocegenomics approach (described in PCT/EP2004/012572, “USE OF ORGANIC COMPOUND”, filed Nov. 11, 2004), the GPA071 peptide was injected into mice and gene expression profiling on many organs performed. Surprisingly, the GPA071 peptide showed significant effects on regulation of gene expression in the liver. The genes affected are members of the integrin signalling pathway, wnt pathway and PTEN pathway. See, TABLE 1.

TABLE 1

| Fold | | |
|---|---|---|
| Change | Gene Title | Gene Name |
| 1.33 | Actn1 | actinin, alpha 1 |
| 0.81 | Atf4 | activating transcription factor 4 |
| 0.74 | Atf5 | activating transcription factor 5 |
| 0.78 | Apc | adenomatosis polyposis coli |
| 0.82 | Anxa7 | annexin A7 |
| 1.39 | Catnb | catenin beta |
| 0.57 | Cd151 | CD151 antigen |
| 0.73 | Ccl28 | chemokine (C-C motif) ligand 28 |
| 1.51 | Ccl9 | chemokine (C-C motif) ligand 9 |
| 0.75 | Crsp3 | cofactor required for Sp1 transcriptional activation, subunit 3 |
| 0.68 | Cttn | cortactin |
| 1.23 | Pscd1 | cytohesin 1 (pleckstrin homology, Sec7 and coiled-coil |
| | | domains 1) |
| 0.81 | Dvl1 | dishevelled, dsh homologue 1 (Drosophila) |
| 0.70 | Dst | dystonin |
| 0.83 | FAK | Focal adhesion kinase (PTK2 protein tyrosine kinase 2) |
| 1.30 | Ihh | Indian hedgehog |
| 0.80 | Ikbkg | inhibitor of kappaB kinase gamma |
| 0.75 | Ilk | integrin linked kinase |
| 0.78 | Ick | intestinal cell kinase |
| 1.35 | Lifr | leukaemia inhibitory factor receptor |
| 0.68 | Madh4 | MAD homologue 4 (Drosophila) |
| 0.67 | Mapkapk2 | MAP kinase-activated protein kinase 2 |
| 0.83 | Mknk1 | MAP kinase-interacting serine/threonine kinase 1 |
| 0.78 | Mta1 | metastasis associated 1 |
| 1.22 | BC024131 | metastasis suppressor 1 |
| 0.77 | Mapk8 | mitogen activated protein kinase 8 |
| 0.79 | Map3k12 | mitogen activated protein kinase kinase kinase 12 |
| 0.67 | Prkcl | protein kinase C, lambda |
| 0.67 | Prkcl | protein kinase C, lambda |
| 0.78 | Ppp2r1a | protein phosphatase 2 (formerly 2A), regulatory subunit A (PR |
| | | 65), alpha isoform |
| 1.38 | Arhu | ras homologue gene family, member U |
| 0.68 | 5133400C09Rik | RIKEN cDNA 5133400C09 gene |
| 1.21 | Sstr2 | somatostatin receptor 2 |
| 0.79 | Sos1 | Son of sevenless homologue 1 (Drosophila) |
| 1.29 | Shc1 | src homology 2 domain-containing transforming protein C1 |
| 0.68 | Socs4 | suppressor of cytokine signalling 4 |
| 0.82 | Akt1 | thymoma viral proto-oncogene 1 |
| 1.23 | Tgfa | transforming growth factor alpha |
| 0.65 | Tgm2 | transglutaminase 2, C polypeptide |
| 0.73 | Usf2 | upstream transcription factor 2 |
| 1.41 | Crk | v-crk sarcoma virus CT10 oncogene homologue (avian) |
| 1.41 | Crk | v-crk sarcoma virus CT10 oncogene homologue (avian) |
| 1.36 | Wnt11 | wingless-related MMTV integration site 11 |

The changes in gene expression indicate a positive effect of the GPA071 peptide, and PLA2 generally, on cell proliferation and invasiveness, the gene annotation points at colorectal cancer.

The structure of phospholipase A2 polynucleotides and polypeptides is known. The growing phospholipase A2 superfamily of signal transduction enzymes: Dennis E A, Trends in Biochemical Sciences 22:1 (1997). The peptide sequence of GPA071 isolated from human plasma (Identification number GP_{—}1221076) is AVWQFRKMIKCVIPGSDPFLEYNNYGCYCGLGGSGTPVDELDKCCQTHDNCYDQAKKLDS CKFLLDNPYTHTYSYSCSGSAITCSSKNKECEAFICNCDRNAAICFSKAPYNKAHKNLDTKK YCQS (SEQ ID NO:1). See, PCT/EP2004/007842, “SECRETED POLYPEPTIDE SPECIES REDUCED IN CARDIOVASCULAR DISORDERS” filed Jul. 15, 2004 (incorporated herein by reference). The peptide sequence of a synthesised 124 amino acid a mouse GPA071 is AVWQFRNMIKCTIPGSDPLKDYNNYGCYCGLGGWGTPVDDLDRCCOTHDHCYSQAKKLE SCKFLIDNPYTNTYSYSCSGSEITCSAKNNKCEDFICNCDREAAICFSKVPYNKEYKNLDTGK FC (SEQ ID NO:2).

Based on the findings in this gene expression analysis, phospholipase A2 acts through the activation of the focal adhesion complex and the integrin signalling pathway. This may occur indirectly via an increased release of bile acids, which in turn may activate focal adhesion kinase and the downstream signalling events, resulting in a potential netto pro-proliferative and pro-migratory effect. Debruyne P R et al, Oncogene 21(44): 6740-50 (Oct. 3, 2002). Evidence for a possible link between bile acids and integrin signalling are suggested by Haussinger D et al., Gastroenterology 124(5): 1476-87 (May 2003). However, the changes in gene expression can be due to compensatory effects and cover an anti-proliferative effect (see below). In vivo confirmation experiments, such as those provided herein, are necessary.

Many genes with a proven involvement in colorectal cancer were affected by the injection of GPA071, e.g. beta-catenin (see, Waterman M L, Cancer Metastasis Rev. 23(1-2): 41-52 (January-June 2004)), APC, wnt11 (intestinal cancer). Phospholipase A2 has been linked to colorectal cancer and APC signalling as well, although secreted phospholipase A2 is thought to be protective against colorectal tumours. Kennedy B P et al., Cancer Res. 58(3): 500-3 (Feb. 1, 1998).

Velocegenomics Method

The Velocegenomics method is described in PCT/EP2004/012572, “USE OF ORGANIC COMPOUND”, filed Nov. 11, 2004 (incorporated herein by reference). A peptide (here GPA071) is administered subcutaneously to male C57BL/6 mice for 7 to 14 days at a dose of 300, 600 or 1000 microg/day. At the end of the treatment period samples from all organs are subjected to snap freezing at necropsy and are analyzed with GeneChip® expression profiling.

Total RNA is extracted from these frozen tissues using TRIzol reagent (Life Technologies) according to the manufacturer's instructions. Total RNA is quantified by the absorbance at λ=260 nm (A260nm), and the purity is estimated by the ratio A260 nm/A280nm. Integrity is checked by denaturing gel electrophoresis. RNA is stored at −80° C. until analysis. Good quality total RNA is used to synthesise double-stranded cDNA using the Superscript Choice System (Life Technologies). The cDNA is then in vitro transcribed (MEGAscript™ T7 Kit, Ambion) to form biotin labelled cRNA. Next, 12 to 15 mg of labelled cRNA is hybridised to the Affymetrix Mouse MOE430A expression probe arrays for 16 hours at 45° C. Arrays are then washed according to the EukGE-WS2 protocol (Affymetrix), and stained with 10 mg/ml of streptavidin-phycoerythrin conjugate (Molecular Probes). The signal is antibody-amplified with 2 mg/ml acetylated BSA (Life Technologies), 100 mM MES, 1 M [Na+], 0.05% Tween 20, 0.005 % Antiofoam (Sigma), 0.1 mg/ml goat IgG and 0.5 mg/ml biotinylated antibody and re-stained with the streptavidin solution. After washing, the arrays are scanned twice with the Gene Array® scanner (Affymetrix).

The expression level is estimated by averaging the differences in signal intensity measured by oligonucleotide pairs of a given probe (AvgDiff value). The image acquisition and numerical translation software used for this study is the Affymetrix Microarray Suite version 5 (MAS5). To identify genes that are impacted by treatment, the dataset is initially filtered to exclude in a first wave of analysis genes whose values are systematically in the lower expression ranges where the experimental noise is high (at least an AvgDiff value of 50 in a number of experiments corresponding to the smallest number of replicas of any experimental point). In a second round of selection a threshold t-test p-value (0.05) identifies genes with different values between treated and non-treated based on a two component error model (Global Error Model) and, where possible, with a stepdown correction for multi-hypothesis testing (Benjamini and Hochberg false discovery rate).

The selected genelists are then compared with established genelists for pathways and cellular components using Fisher's exact test. Venn diagrams are used to identify the gene changes that are in common between the different organs. Expression profiles of highly relevant genes are used to find genes with correlated changes at individual experimental points, using several distance metrics (standard, Pearson).

The decision to consider a specific gene relevant is based on a conjunction of numerical changes identified by exploratory filtering and statistical algorithms as described above and the relationship to other modulated genes that point to a common biological theme.

Peptides of the Invention

The term “polypeptide” as used herein, refers to a protein, peptide, oligopeptide or synthetic oligopeptide. These terms are intended to be used interchangeably. Any one of said terms refers to a chain of two or more amino acids which are linked together with peptide or amide bonds, regardless of post-translational modification such as glycosylation or phosphorylation. The polypeptides may also comprise more than one subunit, where each subunit is encoded by a separate DNA sequence.

The polypeptide according to the invention may comprise GPA071 having the amino acid sequence of SEQ ID NO: 1 or SEQ ID NO: 2. A polypeptide of the invention also includes a functionally active polypeptide fragment of the polypeptide of the invention. As shown herein, functional activity can be demonstrated by measuring the activity of the peptide in modulating gene expression in a model system, such as by the Velocegeneomics method. The term “bioactive”, as used herein, refers to a molecule that elicits or affects a biological event. In a preferred embodiment, the level of biological activity of GPA071 or a fragment thereof is measured by detecting the level of expression of one or more genes set forth in TABLE 1. Preferably, the expression of the majority of genes of TABLE 1 is determined. A “bioactive polypeptide” of the invention includes GPA071 and fragments thereof. Also included are homologues which have an amino acid sequence having a percentage of identity of at least 50% to GPA071 and functional activity.

Such polypeptide fragment is meant to be a polypeptide having an amino acid sequence that entirely is the same in part, but not in all, of the amino acid sequence of a polypeptide of the invention. Such polypeptide fragment may be “free-standing,” or may be part of a larger polypeptide of which such polypeptide fragment forms a part or region, most preferably as a single continuous region. A fragment may comprise at least 10 amino acids, preferably at least 15, 20, or 25 amino acids. More preferably a fragment comprises at least 30 amino acids. Another preferred fragment comprises at least 40, 50, 60, 65, 70 or 75 amino acids. Most preferably the fragment comprises 12, 22, 32, 35, 39, 66, 72 or 75 consecutively amino acids of SEQ ID NO:1 or SEQ ID NO:2.

Such polypeptide may also be a fragment such as a proteolytic cleavage product, e.g. generated by proteases such as for example by trypsin. A polypeptide or a polypeptide fragment according to the invention may comprise a C-terminal fragment of SEQ ID NO:1 or SEQ ID NO:2. Such C-terminal fragment may comprise at least 10 amino acids of the C-terminus, preferably at least 20 or 25, even more preferred at least 30 amino acids or at least 65, 70 or 75 amino acids. The at least 10 amino acids may comprise the most C-terminal at least 10 amino acids. The polypeptide may comprise the at least 32, 35, 39, 66, 72 or 75 most C-terminal amino acids of SEQ ID NO:1 or SEQ ID NO:2. A fragment may also comprise an internal fragment o.

The polypeptide may also have an amino acid sequence having a percentage of identity of at least 50%, preferably at least 60%, more preferred at least 70% or 80%, and most preferably at least 90% such as 95%, 97%, or 99% identity with the amino acid sequence of any one of the aforementioned polypeptides such as SEQ ID NO:1 or SEQ ID NO:2.

Amino acid residues are referred to herein by their standard single-letter or three-letter notations: A (Ala) alanine; C (Cys) cysteine; D (Asp) aspartic acid; E (Glu) glutamic acid; F (Phe) phenylalanine; G (Gly) glycine; H (His) histidine; I (lie) isoleucine; K (Lys) lysine; L (Leu) leucine; M (Met) methionine; N (Asn) asparagine; P (Pro) proline; Q (Gln) glutamine; R (Arg) arginine; S (Ser) serine; T (Thr) threonine; V (Val) valine; W (Trp) tryptophan; Y (Tyr) tyrosine.

The term “percentage (%) of identity”, or like term, used in respect of the comparison of a reference sequence and another sequence (i.e. a “candidate” sequence), means that in an optimal alignment between the two sequences, the candidate sequence is identical to the reference sequence in a number of subunit positions equivalent to the indicated percentage, the subunits being nucleotides for polynucleotide comparisons or amino acids for polypeptide comparisons. As used herein, an “optimal alignment” of sequences being compared is one that maximises matches between subunits and minimises the number of gaps employed in constructing an alignment. Percent identities may be determined with commercially available implementations of algorithms described by Needleman & Wunsch, J. Mol. Biol. 48: 443-453 (1970) (“GAP” program of Wisconsin Sequence Analysis Package, Genetics Computer Group, Madison, Wis.). Other software packages in the art for constructing alignments and calculating percentage identity or other measures of similarity include the “BestFit” program, based on the algorithm of Smith & Waterman, Advances in Applied Mathematics 2: 482-489 (1981) (Wisconsin Sequence Analysis Package, Genetics Computer Group, Madison, Wis.). The percentage of identity may also be generated by WU-BLAST-2. Altschul et at., Methods in Enzymology 266: 460-480 (1996). WU-BLAST-2 used several search parameters, most of which are set to the default values. The adjustable parameters are set with the following values: overlap span=1, overlap fraction=0.125, word threshold (T)=11. A % amino acid sequence identity value is determined by the number of matching identical residues divided by the total number of residues in the aligned region. For example, to obtain a polypeptide having an amino acid sequence at least 95% identical to a reference amino acid sequence, up to five percent of the amino acid residues in the reference sequence may be deleted or substituted with another amino acid, or a number of amino acids up to five percent of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence of in one or more contiguous groups with in the references sequence. It is understood that in making comparisons with reference sequences of the invention that candidate sequence may be a component or segment of a larger polypeptide or polynucleotide and that such comparisons for the purpose computing percentage identity is to be carried out with respect to the relevant component or segment.

The invention also includes functionally preserved variants of the polypeptides or polypeptide fragments described herein. Such variants may be made using methods standard in the art, for example, by conservative amino acid substitutions. Typically such substitutions are among Ala, Val, Leu and lie; among Ser and Thr; among the acidic residues Asp and Glu; among Asn and Gin; and among the basic residues Lys and Arg; or aromatic residues Phe and Tyr. Particularly preferred are variants in which several, 5 to 10, 1 to 5, or 2 amino acids are substituted, deleted or added, in any combination.

In various other embodiments, the polypeptide or fragment thereof or polypeptide variant or homologue may be linear or branched, it may comprise modified amino acids, it may be interrupted by non-amino acids, and/or it may be assembled into a complex of more than one polypeptide chain. As is well understood in the art, a polypeptide may be modified naturally or by intervention; for example, disulfide bond formation, glycosylation, lipidation, acetylation, phosphorylation, or any other manipulation or modification, such as conjugation with a labelling component. In some embodiments, polypeptides or polypeptide fragments contain one or more analogues of an amino acid (including, for example, unnatural amino acids, etc.), as well as other modifications known in the art.

A polypeptide or a polypeptide fragment of the invention includes isolated naturally occurring polypeptides. Preferably, such a naturally occurring polypeptide has a frequency in a selected population of at least five percent, and most preferably, of at least ten percent. The selected population may be any recognised population of study in the field of population genetics. Preferably, the selected population is Caucasian, Negroid, or Asian. More preferably, the selected population is French, German, English, Spanish, Swiss, Japanese, Chinese, Korean, Singaporean of Chinese ancestry, Icelandic, North American, Israeli, Arab, Turkish, Greek, Italian, Polish, Pacific Islander, or Indian.

Recombinant Synthesis of Peptides

A polypeptide or fragment thereof of the invention may also include recombinantly produced polypeptides, synthetically produced polypeptides and a combination of such polypeptides of the invention, and fragments thereof. Means for preparing such polypeptides are well understood in the art. For instance, a polynucleotide fragment or a polypeptide of the invention can be isolated from body fluids including, but not limited to, serum, urine, and ascites, or synthesised by chemical or biological methods (for example, cell culture, recombinant gene expression). “Isolated”, if not otherwise specified herein includes the meaning “separated from coexisting material”.

Recombinant polypeptides of the present invention may be prepared by processes well known in the art from genetically engineered host cells comprising expression systems. Accordingly, in a further aspect, the present invention relates to the production of polypeptides by recombinant techniques, to expression systems which comprise a nucleic acid or nucleic acids encoding the polypeptides of the present invention, to host cells which are genetically engineered with such expression systems, and to methods to isolate the polypeptides.

The term “nucleic acid” means natural or semi-synthetic or synthetic or modified nucleic acid molecules. It refers to nucleotide sequences, oligonucleotides or polynucleotides including deoxyribonucleic acid (DNA) and/or ribonucleic acid (RNA) and/or modified nucleotides. These terms are intended to be used interchangeably. RNA may be in the form of a tRNA (transfer RNA), snRNA (small nuclear RNA), rRNA (ribosomal RNA), mRNA (messenger RNA), anti-sense RNA, and ribozymes. DNA may be in form of plasmid DNA, viral DNA, linear DNA, chromosomal or genomic DNA, cDNA, or derivatives of these groups. In addition these DNAs and RNAs may be single, double, triple, or quadruple stranded. The term also includes PNAs (peptide nucleic acids), phosphorothioates, and other variants of the phosphate backbone of native nucleic acids.

“Stringent conditions” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends upon the ability of a denatured nucleic acid to reanneal when complementary strands are present in an environment near but below their melting temperature. The higher the degree of homology between the probe and the hybridisable sequence, the higher the relative temperature which can be used. As a result, it follows that higher relative temperatures would tend to make the reaction conditions more stringent, while lower temperatures less so. Moreover, stringency is also inversely proportional to salt concentrations. “Stringent conditions” are exemplified by reaction conditions characterised by: (1) low ionic strength and high temperature for washing, for example 0.015 M sodium chloride/0.0015 M sodium citrate/0.1% sodium dodecyl sulfate at 50° C.; (2) the use of a denaturing agent, such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C. Alternatively, stringent conditions can be: 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 □g/ml) 0.1% SDS, and 10% dextran sulfate at 42° C., with washes at 42° C. in 0.2×SS (sodium chloride/sodium citrate) and 50% formamide at 55° C., followed by a high-stringency wash consisting of 0.1×SSC containing EDTA at 55° C. For additional details and explanation of stringency of hybridization reactions, see Ausubel et al., Protocols in Molecular Biology (1995).

The polypeptide can be expressed recombinantly in any of a number of expression systems according to methods known in the art. Ausubel et al., editors, Current Protocols in Molecular Biology (John Wiley Sons, New York, 1990). Such expression systems include chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

The expression systems may contain control regions that regulate as well as engender expression. Generally, any system or vector which is able to maintain, propagate or express a nucleic acid to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual, 2nd Ed. (Cold Spring Harbor Laboratory Press, Cold Spring Harbor, N.Y., 1989). In general, DNA is inserted into an appropriate restriction endonuclease site using techniques known in the art.

Vector components generally include, but are not limited to, one or more of an origin of replication, one or more marker genes, an enhancer element, a promoter, a signal or secretion sequence, and a transcription termination sequence:

The expression vector may have two replication systems, thus allowing it to be maintained in two organisms, for example in mammalian or insect cells for expression and in a prokaryotic host for cloning and amplification. Such sequences are well known for a variety of bacteria, yeast strains, and viruses.

Preferably, the expression vector contains a marker gene to allow the selection of transformed host cells. Selection genes are well known in the art and will vary with the host cell used. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients e.g., the D-alanine racemase gene.

Promoter sequences encode either constitutive or inducible promoters. The promoters may be either naturally occurring promoters or hybrid promoters. Hybrid promoters, which combine elements of more than one promoter, are also known in the art, and are useful in the present invention. Further, for integrating expression vectors, the expression vector contains at least one sequence homologous to the host cell genome, and preferably, two homologous sequences which flank the expression construct. The integrating vector may be directed to a specific locus in the host cell by insertion of the appropriate homologous sequence in the vector. Constructs for integrating vectors are well known in the art.

An appropriate secretion signal may be incorporated into the desired polypeptide to allow secretion of the polypeptide into the lumen of the endoplasmic reticulum, the periplasmic space or the extracellular environment. These signals may be endogenous to the polypeptide or they may be heterologous signals. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, the alpha factor leader (including Saccharomyces and Kluyveromyces α-factor leaders). In mammalian cell expression systems, mammalian signal sequences from secreted polypeptides of the same or related species, as well as viral secretory leaders may be used to direct secretion of the peptide, variants or homologues thereof. Appropriate host cells include yeast, bacteria, archebacteria, fungi, and insect and animal cells, including mammalian cells, for example primary cells, including but not limited to stem cells. Representative examples of appropriate hosts include bacterial cells, such as E. coli, Streptococci, Staphylococci, Streptomyces, and Bacillus subtilis; fungal cells, such as Saccharcmyces cerevisiae, other yeast cells or Aspergillus; insect cells such as Drosophila S2 and Spodoptera Sf9 cells; animal cells such as CHO, COS, HeLa, C127, 3T3, BHK, HEK 293 and Bowes melanoma cells; and plant cells.

A host cell strain may be chosen for its ability to modulate the expression of the inserted sequences or to process the expressed polypeptide in the desired fashion. Such modifications of the polypeptide include, but are not limited to, acetylation, carboxylation, glycosylation, phosphorylation, lipidation and acylation. Post-translational processing, which cleaves a “prepro” form of the polypeptide, may also be important for correct insertion, folding and/or function.

Transformed host cells include, but are not limited to, microorganisms such as bacteria transformed with recombinant bacteriophage, plasmid or cosmic DNA expression vectors, yeast transformed with yeast expression vectors, and insect cells infected with a recombinant insect virus (such as baculovirus), and mammalian expression systems.

The appropriate conditions for expression of peptides will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used together with insect cells are lytic viruses, and thus harvest time selection can be crucial for product yield.

The desired GPA071 peptide fragment may be produced recombinantly not only directly, but also as a fusion polypeptide with a heterologous polypeptide. Such heterologous polypeptide is generally placed at the amino- or carboxyl-terminus of a GPA071 peptide or of a fragment and may provide for an epitope tag to which an anti-tag antibody can selectively bind. Accordingly, such epitope tag enables a peptide or a fragment thereof to be readily purified by using an anti-tag antibody or another type of affinity matrix that binds to the epitope tag. Examples of epitope tags are 6xHis or c-myc tag. Alternatively a GPA071 peptide or a fragment thereof may be expressed in the form of e.g. a GST-fusion protein. Appropriate constructs are generally known in the art and are available from commercial suppliers such as Invitrogen (San Diego, Calif., USA), Stratagene (La Jolla, Calif., USA), Gibco BRL (Rockville, Md., USA) or Clontech (Palo Alto, Calif., USA).

Evaluation of Gene Expression

Gene expression may be evaluated in a sample directly, for example, by standard techniques known to those of skill in the art, e.g., Southern blotting for DNA detection, Northern blotting to determine the transcription of mRNA, dot blotting (DNA or RNA), or in situ hybridization, using an appropriately labelled probe, based on the sequences provided herein. Alternatively, antibodies may be used in assays for detection of nucleic acids, such as specific duplexes, including DNA duplexes, RNA duplexes, and DNA-RNA hybrid duplexes or DNA-protein duplexes. Such antibodies may be labelled and the assay carried out where the duplex is bound to a surface, so that upon the formation of duplex on the surface, the presence of antibody bound to the duplex can be detected. Gene expression, alternatively, may be measured by immunohistochemical staining of cells or tissue sections and assay of cell culture or body fluids, to directly evaluate the expression of a GPA071 peptide or fragment. Antibodies useful for such immunological assays may be either monoclonal or polyclonal, and may be prepared against a native sequence phospholipase A2 or GPA071 fragments based on the DNA sequences provided herein.

Purification of Expressed Protein

Expressed GPA71 or GPA71 fragments may be purified or isolated after expression, using any of a variety of methods known to those skilled in the art. The appropriate technique will vary depending upon the way of expression of GPA71 or GPA71 fragment. The polypeptide may for example be recovered from culture medium in the form of a secreted protein or from host cell lysates. Cells can be disrupted by various physical or chemical means, such as freeze-thaw cycling, sonication, mechanical disruption, or by use of cell lysing agents, whereas membrane-bound polypeptides may be released from the membrane using a suitable detergent solution (e.g. Triton-X 100) or by enzymatic cleavage. The appropriate technique for polypeptide purification or isolation will also vary depending upon what other components are present in the sample. The degree of purification necessary will also vary depending on the use of GPA71 or a fragment thereof. Contaminant components that are removed by isolation or purification are materials that would typically interfere with diagnostic or therapeutic uses for the polypeptide, and may include enzymes, hormones, and other solutes. The purification step(s) selected will depend, for example, on the nature of the production process used and the particular fragment produced.

Ordinarily, isolated GPA071 or a fragment thereof will be prepared by at least one purification step. Well-known methods for purification include ammonium sulfate or ethanol precipitation, acid extraction, anion or cation exchange chromatography, phosphocellulose chromatography, hydrophobic interaction chromatography, high performance liquid chromatography, hydroxylapatite chromatography and lectin chromatography. Most preferably, affinity chromatography is employed for purification. Ultrafiltration and dialysis techniques, in conjunction with protein concentration, are also useful. See, for example, Scopes R, Protein Puritication (Springer-Verlag, New York, N.Y., 1982). Well-known techniques for refolding proteins may be employed to regenerate active conformation when the polypeptide is denatured during isolation and or purification

Labelling of Expressed Polypeptide

The nucleic acids, proteins and antibodies of the invention may be labelled. By labelled herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) coloured or fluorescent dyes. The labels may be incorporated into the compound at any position that does not interfere with the biological activity or characteristic of the compound which is being detected.

Chemical Manufacture of Fragments

Polypeptides or fragments thereof may be produced not only by recombinant methods, but also by using chemical methods well known in the art. Solid phase peptide synthesis may be carried out in a batchwise or continuous flow process which sequentially adds alpha-amino- and side chain-protected amino acid residues to an insoluble polymeric support via a linker group. A linker group such as methylamine-derivatised polyethylene glycol is attached to poly(styrene-co-divinylbenzene) to form the support resin. The amino acid residues are N^{alpha}-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesised by sequential deprotection, coupling of derivatised amino acids, and washing with dichloromethane and/or N, N-dimethylformamide. The peptide is cleaved between the peptide carboxy terminus and the linker group to yield a peptide acid or amide. Novabiochem 1997/98 Catalog and Peptide Synthesis Handbook (San Diego, Calif.) pp. S1-S20. Automated synthesis may also be carried out on machines such as the ABI 431A peptide synthesiser (Applied Biosystems). A polypeptide or a fragment thereof may be purified by preparative high performance liquid chromatography and its composition confirmed by amino acid analysis or by sequencing (Creighton T. E. Proteins, Structures and Molecular Properties (W H Freeman, New York N.Y., 1984)

Variants of the natural polypeptide may be desirable in a variety of circumstances. For example, undesirable side effects might be reduced by certain variants, particularly if the side effect activity is associated with a different part of the polypeptide from that of the desired activity. In some expression systems, the native polypeptide may be susceptible to degradation by proteases. In such cases, selected substitutions and/or deletions of amino acids which change the susceptible sequences can significantly enhance yields. Variants may also increase yields in purification procedures and/or increase shelf lives of proteins by eliminating amino acids susceptible to oxidation, acylation, alkylation, or other chemical modifications. Preferably, such variants include alterations that are conformationally neutral, i.e. they are designed to produce minimal changes in the tertiary structure of the variant polypeptides as compared to the native polypeptide, and (ii) antigenically neutral, i.e. they are designed to produce minimal changes in the antigenic determinants of the variant polypeptides as compared to the native polypeptide.

Uses of the Peptides of the Invention

The aforementioned polypeptides may according to the invention be used for the manufacture of a medicament for use in the treatment of a disease or condition associated with excessive cell proliferation, particularly cancer, more particularly colorectal cancer.

A further aspect of the invention provides a method for the treatment of a disease or condition associated with excessive cell proliferation is provided said method comprises administering an effective amount of a polypeptide to a mammal including a human suffering from the disease or condition, wherein the polypeptide is selected from the groups consisting of a) GPA71 (SEQ. ID NO:1 or SEQ ID NO:2) or a fragment of GPA71; b) a bioactive polypeptide having a percentage of identity of at least 50% with the amino acid sequence of any one of the polypeptides of (a); or c) a bioactive variant of any one of the polypeptides of (a) or (b). Accordingly, a polypeptide as described above may be administered. The polypeptide preferably comprises GPA71, or a fragment thereof.

“Mammal” for purposes of treatment refers to any animal classified as a mammal, including humans, domestic and from animals, and zoo, sports, or pet animals, such as dogs, horses, cats, sheep, pigs, cattle, etc. Preferably, the mammal is human.

The term “treatment” refers to both therapeutic treatment and prophylactic or preventative measures. Those in need of treatment include those already with the disorder as well as those in which the disorder is to be prevented.

A “disease” or a “condition” is any condition that would benefit from treatment with CPA71 or a fragment of GPA71 as defined above and further below. This includes both chronic and acute diseases and conditions, as well as those pathological conditions which predispose to the disease or condition in question.

Non-limiting examples of diseases or conditions to be treated herein include any condition which results from excessive cell proliferation, particularly cancer, and more particularly colorectal cancer. Examples of hyperproliferative diseases include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Other hyperproliferative diseases, disorders, and/or conditions include, but are not limited to: hypergammaglobulinaemia, lymphoproliferative diseases, disorders, and/or conditions, paraproteinaemias, purpura, sarcoidosis, Sezary Syndrome, Waldenstron's Macroglobulinemia, Gaucher's Disease, histiocytosis. Other hyperproliferative diseases are known to those of skill in the medical art.

In another aspect of the invention, GPA71 or a fragment of GPA71 is provided as suitable therapeutics for the treatment of a disease or condition associated with excessive cell proliferation comprising administering an effective amount of GPA71 or a fragment thereof to a mammal, including a human, suffering from said disease.

The pharmaceutical composition may be used in the foregoing methods of treatment. Such compositions are preferably sterile and contain an effective amount of GPA71 or a fragment thereof or a nucleic acid encoding the polypeptide or fragment for inducing the desired response in a unit of weight or volume suitable for administration to a patient.

An “effective amount” of GPA71 or fragment thereof, compound, or pharmaceutical composition is an amount sufficient to effect beneficial or desired results including clinical results such as a cell proliferative disease. Such amounts will also depend on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the medical art and can be addressed with no more than routine experimentation.

An effective amount can be administered in one or more administrations and may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An effective amount of GPA71 or GPA71 fragment or the pharmaceutical composition comprising the polypeptide of the invention, alone or in conjunction with another drug, compound, or pharmaceutical composition can be administered by any conventional route, including injection or by gradual infusion over time. The administration may, for example, be oral, intravenous, intraperitoneal, intramuscular, intracavity, subcutaneous, topical or transdermal.

When administered, the pharmaceutical composition of the present invention is administered in pharmaceutically acceptable preparations. The term “pharmaceutically-acceptable carrier” as used herein means one or more compatible solid or liquid fillers, diluents or encapsulating substances which are suitable for administration into a mammal including humans. The term “carrier” denotes an organic or inorganic ingredient, natural or synthetic, with which the active ingredient is combined to facilitate the application.

The term “pharmaceutically acceptable” means a non-toxic material that does not interfere with the effectiveness of the biological activity of the active ingredients. Such preparations may routinely contain pharmaceutically acceptable concentrations of salts, buffering agents, preservatives, compatible carriers, supplementary immune potentiating agents such as adjuvants and cytokines and optionally other therapeutic agents, such as chemotherapeutic agents.

When used in medicine, the salts should be pharmaceutically acceptable, but non-pharmaceutically acceptable salts may conveniently be used to prepare pharmaceutically-acceptable salts thereof and are not excluded from the scope of the invention.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The doses of polypeptide or nucleic acid encoding said polypeptide administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localised delivery route) may be employed to the extent that patient tolerance permits.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, an elixir or an emulsion.

Compositions suitable for parenteral administration conveniently comprise a sterile aqueous or non-aqueous preparation of a polypeptide or nucleic acid encoding the polypeptide, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences (Mack Publishing Co., Easton, Pa.)

Another aspect of the invention provides a method for the prognosis or diagnosis of a disease or condition associated with excessive cell proliferation comprising detecting the level of GPA71 or a fragment thereof in a biological sample taken from a subject to be diagnosed, wherein an altered level is indicative of a disease or condition associated with excessive cell proliferation. One embodiment of the invention provides a method for the prognosis or diagnosis of a disease or condition associated with excessive cell proliferation in a subject comprising the steps of (i) detecting the level of GPA71 or a fragment thereof in a biological sample obtained from the subject to provide a first value; and step (ii) comparing the first value with a level of GPA71 or fragment thereof from a disease- or condition-free subject, wherein an alteration in the level in the biological sample from the subject compared to the level of GPA71 or fragment thereof in the sample from the disease-free subject is indicative of the subject being predisposed to or having a disease or condition associated with excessive cell proliferation.

Such biological sample includes a blood, plasma or tissue sample. Suitable tissue samples include whole blood, semen, saliva, tears, urine, faecal material, sweat, buccal smears, skin, and biopsies of specific organ tissues, such as muscle, brain or nerve tissue and hair. Most preferably, a suitable biological sample comprises blood or plasma. Tissue samples also include cells and cell types isolated from such biological sample. An alteration in the level of GPA71 or a fragment of thereof may according to a preferred embodiment of the invention comprise an increased plasma level of GPA71 or a fragment thereof within the context of the present invention is relative to the GPA71 or a GPA71 fragment plasma level as found in individuals which do not suffer of a disease or condition associated with excessive cell proliferation. The increase is preferably at least 1.2 fold, more preferably at least 1.5 fold, 2 fold, 3 fold, 5 fold or 10 fold.

According to a further aspect, the present invention provides a method for the prognosis or diagnosis of a disease or condition associated with excessive cell proliferation comprising i) detecting a level of expression of at least one gene identified in Table 1 in a sample of a suitable tissue obtained from the subject to provide a first value; and ii) comparing the first value with a level of expression of said gene from a disease-free subject, wherein a greater or smaller expression level in the subject sample as compared to the sample from the disease-free subject is indicative of the subject being predisposed to or having a disease or condition associated with excessive cell proliferation. Gene expression may be detected on mRNA or protein level. Suitable tissues include, but are not limited to liver, heart, intestines such as duodenum, spleen, bone marrow. The mRNA expression level may be detected by any suitable technique, such as for instance Microarray analysis, Northern blot analysis, reverse transcription PCR and real time quantitative PCR. Likewise, the protein level may be detected by any suitable technique, such as for instance through western blotting by utilizing a labelled probe specific for the protein.

In a preferred embodiment of the above aspects, the gene(s) are selected from the gene(s) set forth in TABLE 1 which are upregulated or downregulated. Preferably, the gene(s) are selected from the gene(s) set forth in TABLE 1 which are upregulated 1.2 fold or more, 1.3 fold or more, or 1.5, 1.7, 1.8, 1.9 fold or more; or from the gene(s) selected from the genes set forth in TABLE 1 are downregulated 0.8 fold or less, 0.7 fold or less, or 0.6 fold or less. In yet another preferred embodiment of the above aspects, the expression of at least 1, 2, 3, 4, 5, 10, 20, 30, is measured. In another embodiment of the invention, the expression of at least 40, 50, or 60 genes selected from TABLE 1 are measured. A still further embodiment of the invention provides that the expression of a majority of the genes selected from Table 1 is determined such as the expression of at least 65, 70, 80, 90, 100, or at least 110 or the expression of all genes of Table 1 is determined.

A still further aspect of the present invention provides a method of identifying a modulator of a disease or condition associated with excessive cell proliferation comprising the steps of i) contacting a test compound with GPA71 or a fragment of thereof, under sample conditions permissive for at least one biological activity of GPA71 or GPA71 fragment; ii) determining the level of said at least one GPA71/GPA71 fragment biological activity; iii) comparing said level to that of a control sample lacking said test compound. In a preferred embodiment said test compound, which causes said level to change, is selected for further testing as a GPA71 or GPA71 fragment modulator for the prophylactic and/or therapeutic treatment of a disease or condition associated with excessive cell proliferation. In a preferred embodiment, the level of biological activity of GPA71 or of GPA71 fragment is measured by detecting the level of expression of one or more or a of plurality of genes such as at least 61, 70 or 100 genes set forth in Table 1.

Examples of a modulator of a disease or condition associated with excessive cell proliferation include, but are not limited to, antisense nucleotides, ribozymes, double-stranded RNAs and antagonists.

The term “double-stranded RNA”, i.e., sense-antisense RNA, corresponding to at least one nucleic acid encoding a polypeptide of the invention, can also be utilised to interfere with expression of at least one of the disclosed genes. Interference with the function and expression of endogenous genes by double-stranded RNA has been shown in various organisms such as C. elegans as described, e.g., in Fire et al., Nature 391: 806-811 (1998); Drosophilia as described, e.g., in Kennerdell et al., Cell 95(7): 1017-1026 (1998); and mouse embryos as described, e.g., in Wianni et al, Nat. Cell Biol. 2(2): 70-75 (2000). Such double-stranded RNA can be synthesised by in vitro transcription of single-stranded PNA read from both directions of a template and in vitro annealing of sense and antisense RNA strands. Double-stranded RNA can also be synthesised from a cDNA vector construct in which the gene of interest is cloned in opposing orientations separated by an inverted repeat. Following cell transfection, the RNA is transcribed and the complementary strands reanneal.

The term “antagonist” refers to a molecule which, when bound to a polypeptide of the inventions or a fragment thereof reduces or inhibits at least one biological activity of said polypeptide. Antagonists can include, but are not limited to, peptides, proteins, carbohydrates, and small molecules.

In a particularly useful embodiment, the antagonist is an antibody specific for phospholipase A2. The antibody may also be conjugated to a reagent such as a chemotherapeutic, radionuclide, ricin A chain, cholera toxin, pertussis toxin, etc., and serve as a target agent.

In yet another embodiment, the antagonist useful as a therapeutic for treating a disease or condition associated with excessive cell proliferation, such as for example cancer, more particularly colorectal cancer.

The term “isolated” nucleic acid molecule means that the nucleic acid molecule is removed from its original environment (e.g., the natural environment if it is naturally occurring). For example, a naturally occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to decrease the transcription/translation GPA71 or a fragment thereof.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat a disease or condition associated with excessive cell proliferation.

The determination of a therapeutically effective amount is well within the capability of those skilled in the art. For any therapeutic, the therapeutically effective dose can be estimated initially either in cell culture assays or in animal models, usually mice, rabbits, dogs, or pigs. The animal model may also be used to determine the appropriate concentration range and route of administration. Such information can then be used to determine useful doses and routes for administration in humans.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a subject alone, or in combination with other agents, drugs or hormones.

All references cited herein are incorporated herein by reference in their entirety and for all purposes to the same extent as if each individual publication, patent, or patent application was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

What is claimed is:

1. Use of a GPA071 polypeptide for the manufacture of a medicament for use in the treatment of a disease or condition associated with excessive cell proliferation, wherein the polypeptide comprises a peptide having a sequence selected from SEQ ID NO:1, SEQ ID NO:2 or a bioactive fragment thereof.

2. Use of a polypeptide according to claim 1, wherein the disease or condition associated with excessive cell proliferation is cancer.

3. Use of a polypeptide according to claim 2, wherein the disease or condition associated with excessive cell proliferation is colorectal cancer.

4. A method for the treatment of a disease or condition associated with excessive cell proliferation comprising administering an effective amount of a polypeptide comprising a peptide having a sequence selected from SEQ ID NO:1, SEQ ID NO:2 or a bioactive fragment thereof to a mammal having a disease or condition associated with excessive cell proliferation.

5. The method of claim 4, wherein the disease or condition associated with excessive cell proliferation is cancer.

6. The method of claim 4, wherein the disease or condition associated with excessive cell proliferation is colorectal cancer.

7. The method of claim 4, in which the effective amount of the polypeptide is administered intravenously, intramuscularly, subcutaneously, orally or topically.

8. A method for the prognosis or diagnosis of a disease or condition associated with excessive cell proliferation in a subject comprising the steps of:
(i) detecting the level of GPA71 or a fragment thereof in a biological sample obtained from the subject to provide a first value; and
(ii) comparing the first value with a level of GPA71 or fragment thereof from a disease- or condition-free subject, wherein an alteration in the level in the biological sample from the subject compared to the level of GPA71 or fragment thereof in the sample from the disease-free subject is indicative of the subject being predisposed to or having a disease or condition associated with excessive cell proliferation.

9. The method of claim 8, wherein the biological sample is plasma.

10. The method of claim 8, wherein the level of expression of one or more genes identified in TABLE 1 is detected.

11. The method of claim 8, wherein the level of expression of a majority of genes identified in TABLE 1 is detected.

12. A method of identifying a modulator of a disease or condition associated with excessive cell proliferation comprising the steps of:
(i) contacting a test compound with GPA71 or a fragment thereof under sample conditions permissive for at least one biological activity of GPA71 or GPA71 fragment;
(ii) determining the level of said at least one biological activity of GPA71 or GPA71 fragment;
(iii) comparing said level to that of a control sample lacking said test compound; and
(iv) selecting a test compound which causes said level to change for further testing as a GPA71 modulator for the prophylactic and/or therapeutic treatment of a disease or condition associated with excessive cell proliferation.

13. The method according to claim 12, wherein the level of biological activity of GPA71 or a GPA71 fragment is measured by determining the level of expression of one or more genes set forth in TABLE 1.

14. The method according to claim 12, wherein the level of expression of a majority of genes identified in TABLE 1 is detected.
